(19) United States
(12) Patent Application Publication
(10) Pub. No.: US 2005/0178393 A1
(43) Pub. Date: Aug. 18, 2005

(54) Hip reduction device

(76) Inventor: Burton Bentley II, Tucson, AZ (US)

Correspondence Address: LEWIS AND ROCA LLP, 40 NORTH CENTRAL AVENUE, PHOENIX, AZ, 85004, US

(21) Appl. No.: 10/782,605

(22) Filed: Feb. 18, 2004

(51) Int. Cl. A47C 17/86; A61F 13/06

(52) U.S. Cl. 128/892

(57) ABSTRACT

A device to assist a physician in treating a dislocated hip is disclosed. A hip reduction device comprises a base, a powered vertical lifter attached to the base, a transverse support arm attached to the vertical lifter, and a leg sleeve attached to the transverse support arm. In addition the hip reduction device includes a pelvic belt attached to a patient's hospital bed with which to secure the patient to the hospital bed. The operation of the hip reduction device mechanically mimics the Allis maneuver that a physician may use to manually reduce a dislocated hip. A patient suffering from a dislocated hip is secured to a hospital bed with a pelvic belt. The patient's leg is then placed in the leg sleeve. Raising the vertical lifter raises the transverse support arm, and thus raises the patient leg. In this way sufficient mechanical force is generated on the patient's leg so that a dislocated hip is reduced to its normal position.

### BACKGROUND OF THE INVENTION

1. Field of the Invention

This invention relates to the facilitation of hip reduction following hip dislocation. More particularly, the present invention describes a device and provides a method that allows a physician to effect reduction of a patient's dislocated hip joint without exerting significant manual effort or being in an awkward, unstable position.

2. Description of the Related Art

The dislocated hip is a common joint dislocation encountered by emergency physicians and orthopedic surgeons. Hip dislocations are especially prevalent among elderly patients fitted with artificial hip joints. To reduce a dislocated hip, the practice among treating physicians is to do so manually. The procedure to reduce a dislocated hip is called an Allis maneuver. The Allis maneuver is at present a preferred method of care and a defined standard of treatment for orthopedic and emergency physicians.

The Allis maneuver is basically a manual procedure in that the treating physician uses his or her personal force to effect the relocation. In this procedure, the physician must exert a significant amount of energy to produce the large force that is required to perform the maneuver. The application of this large force places a substantial strain on the physician's back. In addition, in performing this maneuver the physician is frequently required to stand atop the patient's bed to gain mechanical advantage. This practice of the physician positioning himself atop the patient places the doctor and patient in an awkward and potentially unstable position. As such, a need has been identified in the art for a device to assist physicians in applying the force necessary to effect the reduction of a dislocated hip.

In anatomic terms, the hip is a ball-and-socket joint stabilized by strong ligamentous and muscular attachments. Dislocation of a native hip joint occurs when the femoral head is displaced from the acetabulum. Similarly, dislocation of a prosthetic, bipolar hip joint occurs when the prosthetic femoral component is displaced from the acetabular cup. Strong muscular and ligamentous attachments surround the hip joint and combine to make the hip an extremely stable joint. This environment of strong muscles and ligaments also work against the treating physician when he or she attempts to overcome their resistance in manually reducing a hip dislocation. Thus it is that a considerable force is often needed to effect the hip reduction. This required level of force can be a challenge for some physicians and can also lead to the patient and physician entwined in tense positions that are awkward and dangerous.

As previously stated, the Allis maneuver is currently the standard of care for hip reduction. During the Allis maneuver, the patient typically lies supine on the examining bed with the hip and knee joints flexed to 90 degrees. An assistant then stabilizes the patient's pelvis by applying downward pressure. Standing over the patient, the treating physician typically directs upward traction indirectly on the femur by pulling the knee upward in a direction nearly perpendicular to the patient. The physician is thereby able to draw the femoral head, or prosthetic ball, anteriorly into the hip joint. Occasionally, slight internal or external rotation of the femur may also be required. A palpable jolt, with or without an audible click, indicates that the ball has re-entered the socket and that the hip joint has been successfully reduced.

Standing over the patient in a bent-over position allows the physician to drape the patient's flexed knee over the physician's flexed elbow. This enables the physician to exert the necessary upward tractional force, but it is an awkward and unstable practice. In addition, this action places a tremendous amount of force on the physician's back, leaving the physician himself susceptible to injury.

Accordingly, it has been recognized that there is a need for a mechanical device that is capable of exerting the necessary upward tractional force on a patient during reduction of a hip dislocation. The mechanical device would permit treatment of the dislocated hip in a way that the treating physician is not required to stand in an unstable position over the patient and exert a large force manually. The hip reduction device should be able to exert the proper force, but be manually operable and give flexibility to the physician. The device should be adjustable to accommodate a wide range of patient physiognomies. In addition, the device should be compatible with the types of beds on which hip reduction procedures are performed, as well as typical hospital equipment and architecture.

### SUMMARY OF THE INVENTION

The present invention is directed to a device that satisfies one or more of the above identified needs to facilitate hip reduction following hip dislocation. In one embodiment, a hip reduction device having features of the present invention comprises a base, a powered vertical lifter that is attached to the base, a transverse support arm that is attached to the powered vertical lifter, a leg sleeve that is attached to the transverse support arm, and a pelvic belt.

The pelvic belt, attached to the patient's bed, is fastened around the patient's pelvis to hold the pelvis down. The padded, contoured leg sleeve is attached around the patient's lower extremity both above and below the patient's knee. The sleeve hangs from the transverse support arm above the patient, and it is detachable from the arm.

The transverse arm rotates freely about the vertical lifter. The vertical lifter is powered, and power application is controlled by the physician. As power is applied, the vertical lifter slowly lifts the transverse support arm and the leg sleeve attached to the patient's leg, applying the necessary upward tractional force on the patient's femur. While the device applies the force, the physician directs the leg movement until the patient's femoral head or prosthetic ball pops back into the hip joint. The power to the lifter is then stopped.

The present invention describes a truly novel device that safely and effectively reduces a dislocated hip, while avoiding the risk of back strain to the physician, since the physician need not apply the significant upward tractional force with his or her own physical strength. Furthermore, danger to both the physician and patient resulting from the physician's awkward and unstable position over the patient is avoided. This represents a vast enhancement over the current practice, which relies on the physician's strength and position relative to the patient. The device mechanically reproduces the standard Allis maneuver as it exerts the necessary upward force that indirectly applies traction to the flexed femur and thereby reduces the dislocated hip.

A first advantage of the hip reduction device is that it can exert an upward tractional force on a human patient so as to effect hip reduction on the patient.

A further advantage of the hip reduction device is that it allows the treating physician to reduce a dislocated hip on a patient without the need for the physician to stand over the patient. Further the device allows patient treatment without the need for the physician to stand, rest, or be supported on the patient's bed.

An additional advantage of the hip reduction device is that it allows a treating physician to reduce a patient's dislocated hip without the need for the physician himself or herself to exert personally a large manual force. Thus the hip reduction device further extends hip reduction treatment procedures to those physicians who may have previously lacked the size, dexterity or robustness to capably carry out the Allis maneuver.

Additionally the hip reduction device may be a moveable device that is adaptable to conventional hospital and emergency room fixtures such as patient beds, operating tables, and other equipment found in hospital rooms.

The hip reduction device is further adaptable to accommodate different patient physiognomies.

Other independent features and advantages of the hip reduction device will become apparent from the following detailed description, taken in conjunction with the accompanying drawings which illustrate, by way of example, the principles of the invention.

### BRIEF DESCRIPTION OF THE DRAWINGS

Characteristics and advantages of a novel hip reduction device according to the present invention will emerge more clearly from the ensuing detailed description, which is provided to give an explanatory and non-limiting example with reference to the accompanying drawings and which illustrate the principles of the invention:

FIG. 1 is a perspective view of a hip reduction device according to the present invention;

FIG. 2 is a detailed view of the pelvic belt assembly of the present invention;

FIG. 3 is a detailed view of the base assembly of the present invention;

FIG. 4 is a detailed view of the powered vertical lifter assembly of the present invention;

FIG. 5 is a detailed view of the transverse support arm assembly according to one embodiment of the present invention;

FIG. 6 is a detailed view of the leg sleeve assembly according to one embodiment of the present invention.

### DETAILED DESCRIPTION OF THE PREFERRED EMBODIMENT

Reference will now be made in detail to exemplary embodiments of the invention, examples of which are illustrated in the accompanying drawings. Wherever possible, the same reference numbers will be used throughout the drawings to refer to the same or like parts.

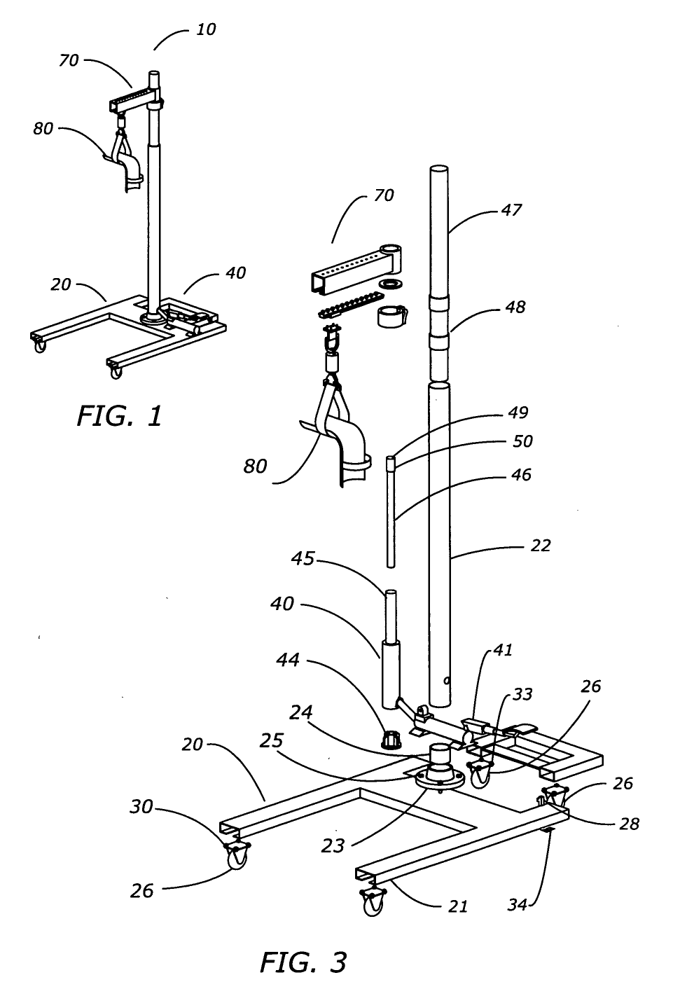

With reference to FIG. 1, one embodiment of the hip reduction device of the present invention is shown in assembled form and is generally indicated with the reference numeral 10. The hip reduction device 10 comprises a base assembly 20, a powered vertical lifter assembly 40, a transverse support arm assembly 70, a leg sleeve assembly 80, and a pelvic belt assembly 60 (not shown in FIG. 1).

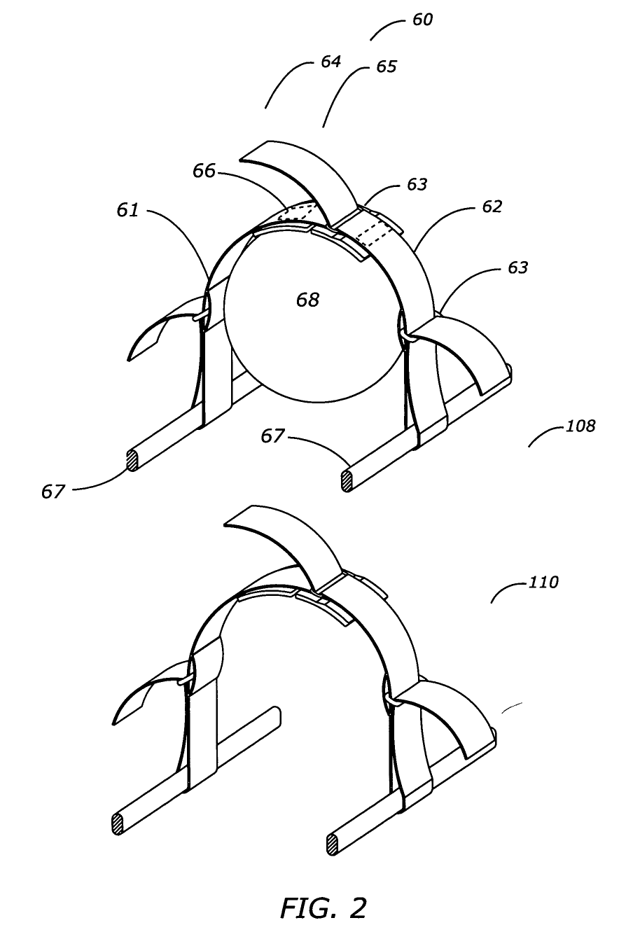

The pelvic belt assembly of the present invention is generally indicated as 60 in FIG. 2. With reference to FIG. 2, the pelvic belt assembly 60 is mounted to the bed or bed rails 67 that are already a part of the hospital bed. In one embodiment, the pelvic belt assembly 60 includes two straps made of, for example, woven thread webbing, a buckle strap 61 and a connector strap 62. On each strap 61 and 62, there is a metal belt loop 63 for attaching the straps to the bed rails 67. The buckle strap 61 has an adjustable buckle 64 attached to the end of the strap, to enable connection with the connector 65 attached to the end of the connector strap 62, thus securing the pelvis of the patient 68. Securing the pelvis of the patient includes securely holding the pelvic/torso area of the human anatomy in order to sufficiently resist the opposing force needed to effect a hip reduction. As an alternative means of connection, the buckle strap 61 could have velcro 66 attached to it in lieu of a buckle, and the connector strap 62 could have a metal belt loop 63 in lieu of the connector. In this alternative, the buckle strap 61 is threaded through the metal belt loop 63 attached to the end of the connector strap 62, and then the buckle strap is secured against itself with the velcro 66. The applicant intends to encompass within the language any means of connecting the straps presently existing or developed in future that would perform the same function.

The base assembly of the present invention in its assembled form is generally indicated as 20 in FIGS. 1 and 3. In one embodiment, with reference to FIG. 3, the base assembly 20 includes a metal base plate 21, to which is attached the shaft 22 that contains the lifting components of the powered vertical lifter 40. The shaft 22 is preferably attached to the base plate 21 using a flange 23 and a bushing 24. The flange 23 is attached to the base 20 plate 21 by fasteners 25. Optionally, to make the hip reduction device mobile, rollers or casters 26 are attached to the base plate 21 with fasteners 30. To enable the operator of the hip reduction device to stabilize it, a rear caster bracket may be attached by hinges to the base plate 21 using fasteners. The rear casters 26 are attached to the rear caster bracket by fasteners. Spring-loaded locking latches 28 may be attached to the base plate 21 by fasteners, such that, when the rear caster bracket is pushed downward, the locking latches 28 lock the bracket in place. In this position, the casters 26 touch the ground and allow the hip reduction device to be rolled on the floor. When the locking latches 28 are released, the rear caster bracket is raised, and the hip reduction device is moved onto pads 34 on the bottom surface of the base plate 21 and is thereby stabilized.

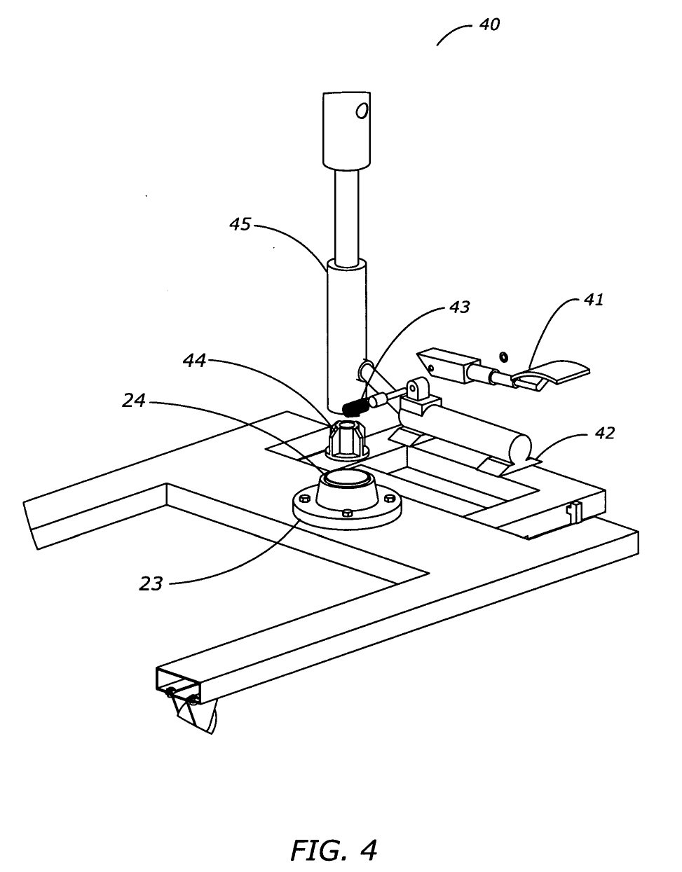

The powered vertical lifter of the present invention in its assembled form is generally indicated as 40 in FIGS. 1, 3 and 4. The vertical lifter can take a variety of different structural configurations. In one embodiment, with reference to FIG. 4, the vertical lifter assembly 40 includes a hydraulic jack with a foot pump 41 attached to the base plate 21 by fasteners 42. Alternatively, a hydraulic hand pump or other means of applying lifting power may be used. Examples of other means to apply lifting force include a mechanic ratcheting assembly and a threaded screw jack. A torsion spring 43 is attached to the pump, as is the lifting cylinder 45, which fits inside the lower shaft 22. The lifting cylinder 45 is attached to the base of the lower shaft 22 by a flange 44. In one embodiment, an extension 46 is attached to the top of the lifting cylinder 45 inside the shaft 22. On the top of the extension 46, a bearing cup 50 is fitted, in which a ball bearing 49 sits. An upper shaft 47 is placed on top of the ball bearing 49. Brass bearings 48 are press fitted inside the lower shaft 22 and attached with fasteners 51 to provide rotational stability to the upper shaft 47. As pressure is applied to the pump 41, the extension 46 and upper shaft 47 are lifted in small, accurate increments. In one embodiment, graduated rule markings, or any similar visual or electronic measuring device, are included along the upper shaft 47 to enable the physician to observe the incremental movement of the vertical lifter. Thus the vertical lifter may also comprise a scale that indicates the lateral position of said lifter assembly in its range of movement.

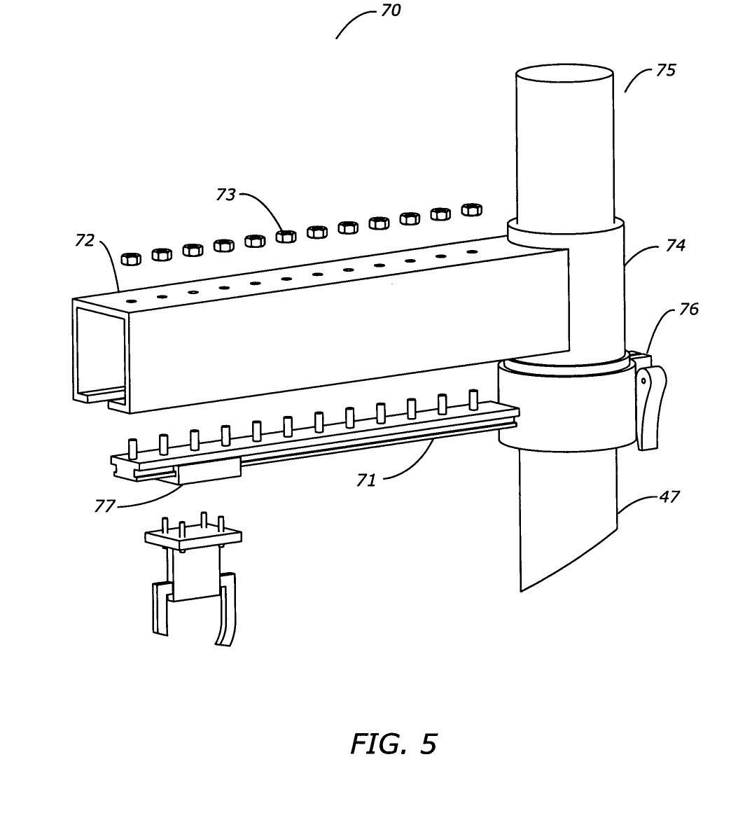

The transverse support arm of the present invention in its assembled form is generally indicated as 70 in FIGS. 1 and 5. The transverse support arm can take a variety of different structural configurations. In a preferred embodiment, the transverse support arm is moveably attached to the vertical lifter assembly 40. In this manner the transverse support arm may be moved in a radial direction with respect to vertical lifter assembly 40 so as to further position the transverse support arm properly over the patient. Preferably, a lock or stop allows the transverse support arm to be secured in a preferred location. Leg sleeve assembly 80 may be affixed to transverse support arm 70 and preferably is affixed in a manner that allows lateral movement of leg sleeve assembly 80 along the length of transverse support arm 70.

In an alternative preferred embodiment, with reference to FIG. 5, a linear rail system 71 is attached to a transverse beam 72 by fasteners 73. The rail system allows the leg sleeve assembly 80 to slide laterally on the linear guide 77. The transverse beam 72 is fixed to a collar 74 as by a weld. The collar is placed over a hollow shaft 75, and the collar 74 and shaft 75 are affixed by a locking collar 76 to the upper shaft 47 of the vertical lifter. A preferred linear rail system is a Thompson linear rail system.

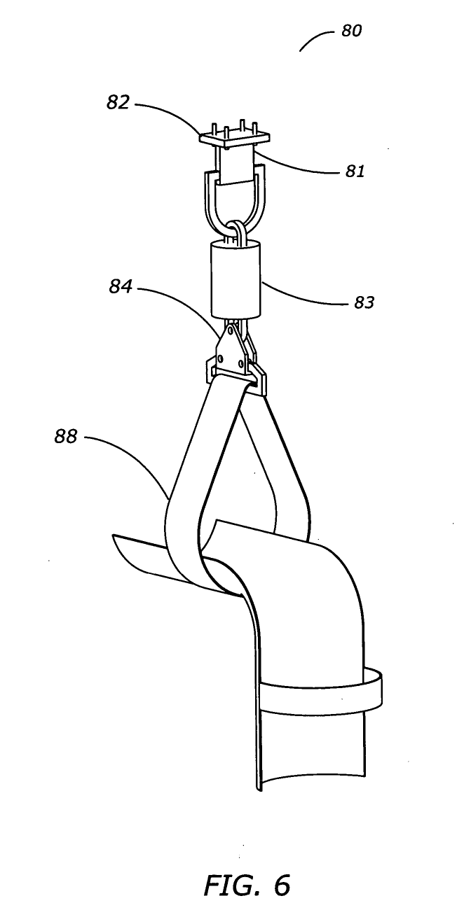

The leg sleeve of the present invention in its assembled form is generally indicated as 80 in FIGS. 1 and 6. In one embodiment, a mounting bracket 81 is attached by fasteners 82 to the linear guide 77 of the transverse support arm assembly. The leg securing device 88 is made of fiberglass, plastic, neoprene, velcro, aluminum, and/or vinyl and is shaped to accommodate a range of sizes of patients' lower extremities. The leg securing device 88 is attached to an adapter 84, which is affixed to a quick release mechanism 83. The quick release mechanism 83 can be easily attached and removed from the mounting bracket 81. In another embodiment, a strain gauge (not shown) is incorporated into the mounting bracket 81 to measure the force exerted on the patient's leg. In yet another embodiment, a ratcheting cable (not shown) is used to attach the leg securing device 88 to the mounting bracket 81.

It is within the scope of the invention herein to modify the hip reduction device from the embodiments described above. For example, a hip reduction device may be constructed that does not include a base. Thus, for example, a vertical lifter assembly could be permanently affixed to a bed or other operating table. Alternatively, a vertical lifter assembly could be affixed to a location in the floor.

In a further alternative embodiment, a transverse support arm assembly may be configured so that the patient's leg rests on top of the transverse support arm assembly. In such an embodiment a saddle or harness affixed to the transverse support arm assembly may secure the patient's leg to this arm. In this configuration, raising the transverse support arm will also raise the patient's leg. In this embodiment a leg sleeve assembly may also be positioned on top of the transverse support arm assembly.

To use the present invention, with reference to FIGS. 1 through 6, first the patient's pelvis is secured to the bed using the pelvic belt assembly 60. By having adjustability, the pelvic belt assembly 60 can accommodate a wide range of patient physiognomies. Then the patient's lower extremity is raised and secured to the leg sleeve 80. By having velcro attachments, the patient's leg can easily be fitted in the leg securing device 88. Through the lateral movement of the linear rail system 71, the leg securing device 88 can be positioned correctly for inserting the patient's leg. A version of the present invention that includes a ratcheting cable (not shown) on the leg sleeve assembly 80 may make securing the patient's lower extremity easier. A version of the present invention that includes a force meter or strain gauge (not shown) on the mounting bracket 81 allows the physician to monitor the amount of force being exerted on the patient's lower extremity. By having rotational freedom, the transverse support arm assembly 70 assists in positioning the leg securing device 88 correctly for coupling with the lower extremity of the patient. This also allows the physician the freedom of movement to manipulate the lower extremity during the application of force, for proper reduction of the patient's femoral head or prosthetic ball into the hip joint.

The base assembly 20 of the present invention provides stability and strength in the device, thus preventing tipping. By having casters 26, the device can be rolled on the floor. However, by having a means to lift the casters, the device can be secured and stabilized at the patient's bed side.

Using the present invention, the physician can remain at the side of the patient's bed, instead of having to stand over the patient and bend over to apply the necessary force. In one embodiment of the present invention, the application of force takes place through the use of a hydraulic foot pump 41. By using a foot pump, the physician has his or her hands free to manipulate the patient's lower extremity during application of the force. The physician can also control the amount of force being applied. The pump calibration provides the proper force and lift increments. The pump allows for the application of a large total upward force in small and accurate increments. Once enough force is applied and the patient's femur has been moved an adequate distance, the physician may gently manipulate the patient's femoral head or prosthetic ball into the hip joint and thus complete the hip reduction.

It is within the scope of this invention that treatment of a patient with a dislocated hip includes both mechanical force applied by the hip reduction device as well as force applied by the treating physician. In addition, the invention includes use of the device where the physician guides or directs the application of the force. Thus in using the device, the device may replace all or part of the force that the physician would otherwise apply manually through the Allis maneuver. One use of the device by the treating physician may be as a supplemental force added to that of the physician. In this manner, when the hip reduction device assists the physician, the physician is relieved of the burden of applying strenuous force directly and manually to the patient's body, and thus the physician will be freed to better direct or guide the force in order to reduce the displaced hip joint with more efficiency. Likewise it is the case that different patients call for a greater or lesser degree of force in effecting a hip reduction. Thus, the hip reduction device may be of particular benefit with respect to those patients for whom a relatively larger degree of force is required to treat the dislocated hip.

While the invention has been described with reference to a preferred embodiment or embodiments, it will be understood by those skilled in the art that various changes may be made and equivalents may be substituted for elements thereof without departing from the scope of the invention. In addition, many modifications may be made to adapt to a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A device for treating a dislocated hip comprising:
a lifter assembly;
a transverse support arm attached to said lifter assembly;
a leg sleeve assembly affixed to said transverse support arm;
means for raising said lifter assembly.

2. The device according to claim 1 further comprising a base wherein said lifter assembly is affixed to said base.

3. The device according to claim 1 further comprising a pelvic belt assembly.

4. The device according to claim 3 wherein said pelvic belt assembly further comprises means for securing said pelvic belt assembly.

5. The device according to claim 4 wherein said means for securing said pelvic belt assembly further comprises at least one strap with at least one end and a buckle affixed to the end of said strap.

6. The device according to claim 1 wherein raising said lifter assembly also raises said transverse support arm.

7. The device according to claim 2 wherein said base further comprises casters to allow movement of said base.

8. A device for assisting a physician in reducing a dislocated hip on a human patient comprising:
a base;
a lifter assembly affixed to said base;
a transverse support arm attached to said lifter assembly;
a leg sleeve assembly affixed to said transverse support arm;
means for raising said lifter assembly.

9. The device according to claim 8 wherein said means for raising said lifter assembly comprises a ratchet.

10. The device according to claim 8 wherein said means for raising said lifter assembly comprises a threaded screw jack.

11. The device according to claim 8 wherein said means for raising said lifter assembly comprises a hydraulic jack.

12. (canceled)

13. The device according to claim 8 further comprising a scale that indicates the lateral position of said lifter assembly.

14. The device according to claim 8 wherein said leg sleeve assembly further comprises a quick release mechanism.

15. A device for treating a dislocated hip comprising:
a base;
a lifter assembly affixed to said base;
a transverse support arm attached to said lifter assembly; and
means for raising said lifter assembly.

16. The device according to claim 15 further comprising a saddle attached to said transverse support arm.

17. The device according to claim 15 wherein said device is used to exert force on a human patient's leg in order to reduce a dislocated hip further comprising a meter for measuring the amount of force exerted on said leg.

18. The device according to claim 15 further comprising a hydraulic foot pump for raising said lifter assembly.

19. A method for treating a dislocated hip of a human patient comprising the steps of:
securing a pelvic belt assembly to a patient;
securing a leg sleeve assembly around a patient's leg;
raising a transverse arm attached to said leg sleeve assembly; and
reducing the dislocated hip joint.

20. The method according to claim 19 further comprising the step of raising a lifter assembly.

21. The method of claim 19 wherein the step of raising a transverse arm attached to said leg sleeve assembly causes a force to be applied to the patient's dislocated hip joint.

22. The method according to claim 19 further comprising the steps of positioning a base and securing a base.

23. The method according to claim 19 further comprising the steps of exerting a force on the patient's dislocated hip and manually directing the force on the patient's hip so as to reduce the dislocated hip.

24. The method according to claim 19 further comprising the step of reading the force applied on a patient from a force meter.

25. A method for treating a dislocated hip of a human patient comprising the steps of:
securing a transverse support arm to the leg of the patient;
raising said transverse arm attached to the patient's leg; and
reducing the dislocated hip of the patient.

26. The method according to claim 25 further comprising the step of securing a pelvic belt assembly to the patient.

27. The method of claim 25 wherein the step of securing a transverse support arm to the leg of the patient further comprises securing the patient's leg to a saddle affixed to said transverse support arm.
